(19) United States
(12) Patent Application Publication
(10) Pub. No.: US 2007/0207182 A1
(43) Pub. Date: Sep. 6, 2007

(54) Medical devices having electrically aligned elongated particles

(76) Inventors: Jan Weber, Maple Grove, MN (US); Liliana Atanasoska, Edina, MN (US)

Correspondence Address: MAYER & WILLIAMS PC, 251 NORTH AVENUE WEST, 2ND FLOOR, WESTFIELD, NJ, 07090, US

(21) Appl. No.: 11/368,738

(22) Filed: Mar. 6, 2006

(51) Int. Cl. A61N 1/00

(52) U.S. Cl. 424/423; 607/2

(57) ABSTRACT

Medical devices configured for implantation or insertion into a subject, which contain one or more particle-containing region that comprises electrically aligned, elongated particles within a matrix. Also described are methods for forming the same.

### FIELD OF THE INVENTION

The present invention relates to medical devices that comprise electrically aligned elongated particles.

### BACKGROUND OF THE INVENTION

If an uncharged, polarizable particle (which may be, for example, an uncharged, polarizable dielectric, semi-conductive or conductive particle) is placed in an electric field, there will be an induced positive charge on one side of the particle and an induced negative charge, of the same magnitude as the induced positive charge, on the other side of the particle. The positive charge will experience a first force; the negative charge will experience a second force in the opposite direction of the first force. In a uniform field, the first and second forces will cancel, and the net force on the particle will be zero. (The same is also true for particles which have permanent dipoles and no net charge.)

In a non-uniform field, however, the electric field will be stronger on one side of the particle and weaker on the other side of the particle. In this case, the forces will not cancel, and there will be a net force on the particle. The lateral motion imparted on uncharged particles as a result of polarization induced by non-uniform electric fields is known as “dielectrophoresis.”

The direction of particle motion is influenced by the polarizability of the surrounding medium. If the suspended particle has a polarizability that is greater than that of the surrounding medium, then the particle is pushed toward the higher electric field region. If the suspended particle has a polarizability that is less than that of the surrounding medium, then the particle is repelled from the higher electric field region. For example, differences in the dielectric constants of metallic and semiconducting single wall carbon nanotubes with respect to a surrounding solvent have been demonstrated to cause opposite movement of metallic nanotubes vs. semiconducting nanotubes along the electric field gradient, allowing them to be separated from one another. See R. Krupke et al., “Separation of Metallic from Semiconducting Single-Walled Carbon Nanotubes,” Science, Vol. 301, 18 Jul. 2003, 344-347.

Moreover, in certain particles, including certain elongated particles such as carbon nanotubes and nanofibers, among others, the dipole moment induced by the electric field, whether uniform or non-uniform, is known to cause a torque on the particle, which tends to align it relative to the electric field. For example, both carbon nanotubes and carbon nanofibers have been used as conductive fillers in epoxy systems (in particular, epoxy systems based on bisphenol-A resin and amine hardener), and AC electric fields have been used to induce the formation of aligned carbon nanotube/nanofiber networks in such systems. DC electric fields were also shown to induce the formation of aligned carbon nanotube networks, although these were less uniform and less aligned than those achieved with the use of AC fields. The quality of the nanotube networks and the resulting bulk conductivity of the composite material was enhanced with increasing field strength. Moreover, electrical anisotropy was observed in the nanofiber-containing composites, and electrical anisotropy was expected to be present in the nanotube-containing composites, based on the observed orientation of the field-induced nanotube networks. For further information, see T. Prasse, “Electric anisotropy of carbon nanofibre/epoxy resin composites due to electric field induced alignment,” Composites Science and Technology 63 (2003) 1835-1841; and C. A. Martin et al., “Electric field-induced aligned multi-wall carbon nanotube networks in epoxy composites,” Polymer 46 (2005) 877-886.

### BRIEF DESCRIPTION OF THE FIGURES

FIGS. 1A and 1B are schematic side and top views, respectively, of an apparatus by which elongated particles may be aligned, in accordance with an embodiment of the present invention.

FIG. 2A is a schematic side view of an apparatus by which elongated particles may be aligned, in accordance with another embodiment of the present invention. FIG. 2B is an end view taken along view v of FIG. 2A. FIG. 2C is a cross sectional view of the device of FIG. 2A taken along the plane corresponding to the line c-c of FIG. 2A.

FIG. 3A is a schematic side view of an apparatus by which elongated particles may be aligned, in accordance with yet another embodiment of the present invention. FIG. 3B is a cross sectional view of the device of FIG. 3A taken along the plane corresponding to the line b-b of FIG. 3A.

FIGS. 4, 5A and 5B are schematic diagrams illustrating a voltage V that is applied to various electrodes over a time t.

FIG. 6A is a schematic side view of an apparatus by which elongated particles may be aligned, in accordance with still another embodiment of the present invention. FIG. 6B is a cross-sectional view taken along the plane corresponding to line b-b of FIG. 6A, and FIG. 6C is a cross-sectional view taken along the plane corresponding to line c-c of FIG. 6A.

### DETAILED DESCRIPTION OF THE INVENTION

According to one aspect of the present invention, medical devices are provided, which include one or more regions in which aligned, elongated particles are present within a within a matrix (also referred to herein as “particle-containing regions”).

Whether or not the elongated particles are aligned can be determined, for example, by microscopic analysis of cross-sections of the particle-containing regions (e.g., using transmission electron microscopy). In some instances, particle alignment can be inferred from significant anisotropy in electrical, mechanical or other physical measurements, for example, exhibiting directional differences of at least 20% to 50% to 100% or more.

Elongated particles may be incorporated into the medical devices of the invention for any of a number of purposes, and the benefits of elongated particles may be further enhanced if the particles are aligned in predetermined directions within the devices. As one example, elongated particles may be incorporated into balloons or balloon coatings to increase strength. In these situations, it may be desirable to align the elongated particles primarily in the direction of the stress vector (e.g., in a circumferential orientation) to further enhance strength. Alternatively, it may be desirable to provide multiple layers containing elongated particles, for example, a first layer having the particles aligned in a direction that is perpendicular to the particles in an adjacent second layer. As another example, conductive elongated particles, such as carbon nanotubes or other conductive filaments, may be introduced to enhance the electrical and/or thermal conductivity of the particle-containing region. Anisotropy of either of these characteristics may be very useful within medical devices. For instance, catheters are known through which one flushes a coolant with the objective of cooling the surrounding tissue in order to minimize tissue damage after a heart attack. In such catheters, it would be desirable to increase the thermal conductivity from the catheter to the surrounding tissue as much as possible at the distal section of the catheter. Carbon nanotubes are known to increase the thermal conductivity of a polymer matrix. When such nanoparticles are aligned in a radial outward direction (e.g., with respect to the catheter shaft), one may achieve enhanced conductivity relative to other spatial distributions.

A. Medical Devices

In certain embodiments, the medical devices in accordance with the present invention are prosthetic devices (i.e., they are artificial substitutes for body parts, such as artificial blood vessels, tissue, etc), whereas in other embodiments they are not. Specific examples of medical devices in accordance with the present invention are therefore many and include medical devices which are adapted for implantation or insertion into a subject, for example, catheters (e.g., renal catheters or vascular catheters such as balloon catheters), guide wires, balloons, filters (e.g., vena cava filters), stents (including coronary vascular stents, cerebral, urethral, ureteral, biliary, tracheal, gastrointestinal and esophageal stents), stent grafts, cerebral aneurysm filler coils (including Guglilmi detachable coils and metal coils), vascular grafts, myocardial plugs, patches, pacemakers and pacemaker leads, heart valves, vascular valves, biopsy devices, patches, and tissue engineering scaffolds for cartilage, bone, skin and other in vivo tissue regeneration, among other devices.

The medical devices of the present invention include medical devices that are used for diagnostics, for systemic treatment, or for the localized treatment of any mammalian tissue or organ. Examples include tumors; organs including the heart, coronary and peripheral vascular system (referred to overall as “the vasculature”), lungs, trachea, esophagus, brain, liver, kidney, bladder, urethra and ureters, eye, intestines, stomach, pancreas, ovary, and prostate; skeletal muscle; smooth muscle; breast; dermal tissue; cartilage; and bone. As used herein, “treatment” refers to the prevention of a disease or condition, the reduction or elimination of symptoms associated with a disease or condition, or the substantial or complete elimination a disease or condition. Typical subjects are mammalian subjects, and more typically human subjects.

In some embodiments, the particle-containing regions for use in the medical devices of the invention correspond to entire medical devices. In other embodiments, the particle-containing regions correspond to one or more portions of a medical device. For instance, the particle-containing regions can be in the form of medical device components, in the form of one or more fibers which are incorporated into a medical device, in the form of one or more layers formed over all or only a portion of an underlying medical device substrate, and so forth. Layers can be provided over an underlying substrate at a variety of locations, and in a variety of shapes or patterns (e.g., in the form of a series of rectangles, stripes, or any other continuous or non-continuous pattern). As used herein a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein.

Substrates for the practice of the present invention include medical device substrates that are incorporated into the finished medical device, as well as substrates that merely acts as templates, but which are not found in the finished device (although a residue of the substrate may remain in certain embodiments, for example, where the substrate is a disintegrable substrate such as a low melting point wax, soluble polymer, etc.).

Suitable substrate materials upon which the particle-containing regions of the present invention may be formed may be selected from a wide variety of materials and include (a) organic materials (e.g., materials containing 50 wt % or more organic species), which may be selected, for instance, from suitable materials listed below for use as matrix materials, and (b) inorganic materials (e.g., materials containing 50 wt % or more inorganic species), which may be selected, for instance, from suitable metallic materials listed below for use as elongated particle materials or from suitable non-metallic inorganic materials listed below for use as matrix materials, among others.

B. Matrix Materials

In general, the aligned, elongated particles are held in place within a matrix. Suitable matrix materials may be selected from a variety of materials, including both inorganic and organic materials.

Inorganic materials may be selected, for instance, from suitable ceramic materials, which may contain, for example, various metal- and non-metal-oxides, various metal- and non-metal-nitrides, various metal- and non-metal-carbides, various metal- and non-metal-borides, various metal- and non-metal-phosphates, and various metal- and non-metal-sulfides, among others. Specific examples may be selected, for example, from suitable inorganic materials containing one or more of the following: metal oxides such as aluminum oxides and transition metal oxides (e.g., oxides of titanium, zirconium, hafnium, tantalum, molybdenum, tungsten, rhenium, and iridium); silicon-based ceramics, such as those containing silicon nitrides, silicon carbides and silicon oxides (sometimes referred to as glass ceramics); calcium phosphate ceramics (e.g., hydroxyapatite); and carbon-based, ceramic-like materials such as carbon nitrides, among many others.

Specific examples of suitable organic materials include polymeric materials (biostable or otherwise) as well as other organic materials. As used herein a “polymeric” material is one that contains polymers, commonly 50 to 75 to 90 to 95 to 97.5 to 99 wt % polymers, or more.

As used herein, “polymers” are molecules containing multiple copies (e.g., on the order of 5 to 10 to 25 to 50 to 100 to 250 to 500 to 1000 or more copies) of one or more constitutional units, commonly referred to as monomers.

Polymers may take on a number of configurations, which may be selected, for example, from cyclic, linear and branched configurations. Branched configurations include star-shaped configurations (e.g., configurations in which three or more chains emanate from a single branch point), comb configurations (e.g., configurations having a main chain and a plurality of side chains), dendritic configurations (e.g., arborescent and hyperbranched polymers), and so forth.

As used herein, “homopolymers” are polymers that contain multiple copies of a single constitutional unit. “Copolymers” are polymers that contain multiple copies of at least two dissimilar constitutional units, examples of which include random, statistical, gradient, periodic (e.g., alternating) and block copolymers.

As used herein, “block copolymers” are copolymers that contain two or more polymer blocks that differ in composition, for instance, because a constitutional unit (i.e., monomer) is found in one polymer block that is not found in another polymer block. As used herein, a “polymer block” is a grouping of constitutional units (e.g., 5 to 10 to 25 to 50 to 100 to 250 to 500 to 1000 or more units). Blocks can be branched or unbranched. Blocks can contain a single type of constitutional unit (also referred to herein as “homopolymeric blocks”) or multiple types of constitutional units (also referred to herein as “copolymeric blocks”) which may be provided, for example, in a random, statistical, gradient, or periodic (e.g., alternating) distribution.

As used herein, a “chain” is a linear (unbranched) grouping of constitutional units.

Organic materials may be selected, for example, from suitable members of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), polycaprolactams and polyacrylamides; resins including alkyl resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropenes) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; various waxes, including low melting point waxes used for dental engineering; as well as blends and further copolymers of the above.

Further organic materials may be selected, for example, from suitable members of the following: (a) homopolymers and copolymers consisting of or containing one or more acrylic acid monomers such as the following: acrylic acid and its salt forms (e.g., potassium acrylate and sodium acrylate); acrylic acid anhydride; acrylic acid esters including alkyl acrylates (e.g., methyl acrylate, ethyl acrylate, propyl acrylate, isopropyl acrylate, butyl acrylate, sec-butyl acrylate, isobutyl acrylate, tert-butyl acrylate, hexyl acrylate, cyclohexyl acrylate, isobornyl acrylate, 2-ethylhexyl acrylate, dodecyl acrylate and hexadecyl acrylate), arylalkyl acrylates (e.g., benzyl acrylate), alkoxyalkyl acrylates (e.g., 2-ethoxyethyl acrylate and 2-methoxyethyl acrylate), halo-alkyl acrylates (e.g., 2,2,2-trifluoroethyl acrylate) and cyano-alkyl acrylates (e.g., 2-cyanoethyl acrylate); acrylic acid amides (e.g., acrylamide, N-isopropylacrylamide and N,N dimethylacrylamide); and other acrylic-acid derivatives (e.g., acrylonitrile); (b) homopolymers and copolymers consisting of or containing one or more methacrylic acid based monomers such as the following: methacrylic acid and its salts (e.g., sodium methacrylate); methacrylic acid anhydride; methacrylic acid esters (methacrylates) including alkyl methacrylates (e.g., methyl methacrylate, ethyl methacrylate, isopropyl methacrylate, butyl methacrylate, isobutyl methacrylate, t-butyl methacrylate, hexyl methacrylate, cyclohexyl methacrylate, 2-ethylhexyl methacrylate, octyl methacrylate, dodecyl methacrylate, hexadecyl methacrylate, octadecyl methacrylate, aromatic methacrylates (e.g., phenyl methacrylate and benzyl methacrylate), hydroxyalkyl methacrylates (e.g., 2-hydroxyethyl methacrylate and 2-hydroxypropyl methacrylate), aminoalkyl methacrylates (e.g., diethylaminoethyl methacrylate and 2-tert-butyl-aminoethyl methacrylate), additional methacrylates (e.g., isobornyl methacrylate and trimethylsilyl methacrylate); and other methacrylic-acid derivatives (e.g., methacrylonitrile); (c) homopolymers and copolymers consisting of or containing one or more vinyl aromatic monomers (i.e., those having aromatic and vinyl moieties) such as the following: unsubstituted vinyl aromatics (e.g., styrene and 2-vinyl naphthalene); vinyl substituted vinyl aromatics (e.g., α-methyl styrene); and ring-substituted vinyl aromatics including ring-alkylated vinyl aromatics (e.g., 3-methylstyrene, 4-methylstyrene, 2,4-dimethylstyrene, 2,5-dimethylstyrene, 3,5-dimethylstyrene, 2,4,6-trimethylstyrene, and 4-tert-butylstyrene), ring-alkoxylated vinyl aromatics (e.g., 4-methoxystyrene and 4-ethoxystyrene), ring-halogenated vinyl aromatics (e.g., 2-chlorostyrene, 3-chlorostyrene, 4-chlorostyrene, 2,6-dichlorostyrene, 4-bromostyrene and 4-fluorostyrene) and ring-ester-substituted vinyl aromatics (e.g., 4-acetoxystyrene); (d) homopolymers and copolymers consisting of or containing one or more vinyl monomers (in addition to vinyl aromatic monomers) such as the following: vinyl alcohol; vinyl esters (e.g., vinyl acetate, vinyl propionate, vinyl benzoate, vinyl 4-tert-butyl benzoate, vinyl cyclohexanoate, vinyl pivalate, vinyl trifluoroacetate and vinyl butyral); vinyl amines (e.g., 2-vinyl pyridine, 4-vinyl pyridine, and vinyl carbazole); vinyl halides (e.g., vinyl chloride and vinyl fluoride); alkyl vinyl ethers (e.g., methyl vinyl ether, ethyl vinyl ether, propyl vinyl ether, butyl vinyl ether, isobutyl vinyl ether, 2-ethylhexyl vinyl ether, dodecyl vinyl ether, tert-butyl vinyl ether and cyclohexyl vinyl ether); and other vinyl compounds (e.g., 1-vinyl-2-pyrrolidone and vinyl ferrocene); (e) homopolymers and copolymers consisting of or containing one or more aromatic monomers (in addition to vinyl aromatic monomers) such as acenaphthalene and indene; (f) homopolymers and copolymers consisting of or containing one or more cyclic ether monomers such as the following: ethylene oxide, trimethylene oxide, propylene oxide, tetrahydrofuran, tetramethylene oxide, methyl glycidyl ether, butyl glycidyl ether, allyl glycidyl ether, epibromohydrin, epichlorohydrin, 1,2-epoxybutane, 1,2-epoxyoctane and 1,2-epoxydecane; (g) homopolymers and copolymers consisting of or containing one or more ester monomers (beyond those ester monomers listed above) such as ethylene malonate; (h) homopolymers and copolymers consisting of or containing one or more alkene monomers such as unsubstituted alkene monomers (e.g., ethylene, propylene, isobutylene, 1-butene, 4-methyl pentene, 1-octene, 1-octadecene, other α-olefins, as well as trans-butadiene, cis-isoprene and trans-isoprene) and substituted alkene monomers such as halogenated alkene monomers (e.g., vinylidene chloride, vinylidene fluoride, cis-chlorobutadiene, trans-chlorobutadiene, and tetrafluoroethylene); (i) homopolymers and copolymers consisting of or containing one or more organo-siloxane monomers such as dimethylsiloxane, diethylsiloxane, methylethylsiloxane, methylphenylsiloxane and diphenylsiloxane, (j) polyamide homopolymers and copolymers formed, for example, from (1) amino acids (i.e., aminocarboxylic acids) such as alpha-aminoacetic acid, epsilon-aminocaproic acid, aspartic acid, glutamic acid, 11-aminoundecanoic acid, beta-benzyl-aspartate, and gamma-benzyl-glutamate, among others, (2) cyclic amides, including lactams, such as glycine anhydride, alpha-pyrrolidone, alpha-piperidone, gamma-butyrolactam, gamma-valerolactam, epsilon-caprolactam, alpha-methylcaprolactam, beta-methylcaprolactam, gamma-methylcaprolactam, delta-methylcaprolactam, epsilon-methylcaprolactam, N-methylcaprolactam, beta,gamma-dimethylcaprolactam, gamma-ethylcaprolactam, gamma-isopropylcaprolactam, epsilon-isopropylcaprolactam, gamma-butylcaprolactam, epsilon-enantholactam, omega-enantholactam, beta-caprylolactam, omega-caprylolactam, and omega-laurolactam, and (3) a combination of one or more diamines and one or more diacids, for example, diamines such as methylene diamine, dimethylene diamine, trimethylene diamine, tetramethylene diamine, pentamethylene diamine, hexamethylene diamine, heptamethylene diamine, octamethylene diamine, nonamethylene diamine, decamethylene diamine, piperazine, diaminocyclohexane, di(aminomethyl)cyclohexane, bis-(4-aminocyclohexyl)methane, bis-(4-amino-1,2-methylcyclohexyl)methane, o-phenylenediamine, m-phenylenediamine, p-phenylenediamine, 4,4′-diaminobiphenyl, tolylenediamine, xylylenediamine, and naphthylenediamine, and diacids such as such as malonic acid, succinic acid, glutaric acid, adipic acid, pimelic acid, suberic acid, azelaic acid, sebacic acid, nonanedicarboxylic acid, decanedicarboxylic acid, undecanedicarboxylic acid, dodecanedicarboxylic acid, hexadecanedicarboxylic acid, thapsic acid, japanic acid, maleic acid, fumaric acid, citraconic acid, diglycollic acid, malic acid, citric acid, phthalic acid, isophthalic acid, terephthalic acid, maleic anhydride, and phthalic anhydride.

In certain embodiments, the matrix materials for use in the present invention are selected, at least in part, based on their associated Tg's (glass transition temperatures). Tg's can generally be measured by differential scanning calorimetry (DSC) (although a few exceptions exist, such as where the Tg of the polymer is above the melting or decomposition temperature of the polymer, etc.). An elevated or “high Tg polymer” is a polymer that displays a glass transition temperature that is above body temperature, more typically from 50° C. to 75° C. to 100° C. to 125° C. or more. A “low Tg polymer” is a polymer that displays a glass transition temperature that is below body temperature, more typically below about 25° C. to 0° C. to −25° C. to −50° C. or less. As used herein, body temperature is 37° C. Typically, polymers displaying low Tg's will be soft and elastic at body temperature, whereas polymers displaying high Tg's will be rigid at body temperature.

In certain embodiments, the matrix materials may include one or more block copolymers, several examples of which are described above. In certain embodiments, the matrix materials may include one or more block copolymers, which in turn contain (a) one or more low T_{g} polymer blocks (designated “L” below) and (b) one or more high T_{g} polymer blocks (designated “H” below), the T_{g} of which, again, can be generally be measured by DSC.

Block copolymer configurations vary widely and include, for example, the following configurations (in which H and L chains are used for illustrative purposes, although other chains having different characteristics can clearly be substituted): (a) block copolymers containing alternating chains of the type (HL)ₘ, L(HL)ₘ and H(LH)ₘ where m is a positive whole number of 1 or more, (b) star block copolymers containing multi-arm geometries such as X(LH)ₙ, and X(HL)ₙ, where n is a positive whole number of 2 or more, and X is a hub species (e.g., an initiator molecule residue, a residue of a molecule to which preformed polymer chains are attached, etc.), and (c) comb copolymers having a L chain backbone and multiple H side chains and those having an H chain backbone and multiple L side chains. Note that it is common to disregard the presence of non-polymeric entities, such as hub species in describing block copolymers, for example, with HL-X-LH being commonly designated as a triblock copolymer HLH.

More specific examples of block copolymers include polyether-polyamide block copolymers which include one or more low T_{g} polyether blocks (i.e., polymer blocks containing multiple C—O—C linkages) and one or more high T_{g} polyamide blocks (i.e., polymer chains containing multiple —NH—CO— linkages). Such block copolymers are commonly used in medical devices, for instance, in balloons, catheters and endoscopes, among others. See, for example, U.S. Pat. No. 5,556,383 to Wang et al. for more information. Many polyether-polyamide block copolymers have excellent mechanical properties, are stable, and are readily processed (e.g., by melt or solution processing).

Further specific examples of polyether-polyamide block copolymers include those containing (a) one or more polyamide homopolymer or copolymer blocks, which may correspond to the polyamide homopolymers and copolymers described above and (b) one or more polyether homopolymer or copolymer blocks, which may contain one or more of the cyclic ether monomers that are described above.

Still further specific examples of polyether-polyamide block copolymers include those containing (a) one or more polyether blocks selected from homopolymer blocks such as polyethylene oxide, poly(trimethylene oxide), poly(propylene oxide) and polytetramethylene oxide, and copolymer blocks such as those containing two or more of the following: ethylene oxide, trimethylene oxide, propylene oxide and polytetramethylene oxide, (b) one or more polyamide blocks selected from nylon homopolymer blocks and copolymer blocks such as nylon 6, nylon 4/6, nylon 6/6, nylon 6/10, nylon 6/12, nylon 11 and nylon 12.

For example, poly(tetramethylene oxide)-nylon-12 block copolymer, is available from Elf Atochem as PEBAX. As indicated above many polyether-polyamide block copolymers, including PEBAX, have excellent mechanical properties, are stable, and are readily processed (e.g., by melt or solution processing). Moreover, many polyether-polyamide block copolymers, including PEBAX, are capable of forming good interfacial contacts with a variety of materials including metals, ceramics and polymers, particularly with polyethers, polyamides, and poly(ether-amide) copolymers.

Specific examples of block copolymers further include polyalkene-poly(vinyl aromatic) block copolymers which include one or more low T_{g} polyalkene blocks and one or more high T_{g} poly(vinyl aromatic) blocks.

Further specific examples of polyalkene-poly(vinyl aromatic) block copolymers include those containing (a) one or more polyalkene homopolymer or copolymer blocks, which may contain one or more of the alkene monomers described above and (b) one or more poly(vinyl aromatic)homopolymer or copolymer blocks, which may contain one or more of the vinyl aromatic monomers described above.

Still further specific examples of polyalkene-poly(vinyl aromatic) block copolymers include those containing (a) one or more polyalkene homopolymer or copolymer blocks, which may contain one or more of ethylene, butylene and isobutylene, and (b) one or more poly(vinyl aromatic)homopolymer or copolymer blocks, which may contain one or more of styrene and alpha-methyl-styrene.

For instance, polyisobutylene-polystyrene block copolymers, including polystyrene-polyisobutylene-polystyrene triblock copolymer (SIBS), are described in U.S. Pat. No. 6,545,097 to Pinchuk et al., which is hereby incorporated by reference in its entirety. These polymers have proven valuable as release polymers in implantable or insertable drug-releasing medical devices, such as stents. These polymers are particularly useful for medical device applications because of their excellent strength as well as their excellent biostability and biocompatibility, particularly within the vasculature.

C. Elongated Particles

Elongated particles for use in the present invention may be formed from a variety of materials and may be provided in a variety of sizes and shapes (e.g., in the form of elongated plates, in the form of solid or hollow filamentous particles having cross-sections of regular or irregular geometry, including cylindrical, tubular, and ribbon-shaped filamentous particles, among many others.)

The elongated particles for use in the present invention are frequently microparticles, meaning that at least one major dimension of the particle (e.g., selected from diameter and length for an elongated particle of circular geometry such as a cylindrical or tubular particle, selected from length, width and thickness for an elongated plate or ribbon, and so forth) is less than 100 microns (μm) in length, for example, ranging from 100 μm to 30 μm to 10 μm to 3 μm to 1000 nm to 300 nm to 100 nm to 30 nm to 10 nm to 3 nm to 1 nm or less. For example, for an elongated plate at least the thickness will fall within this range, for a tubular or cylindrical filamentous particle at least the diameter will fall within this range, for other solid or hollow filamentous particles such as a ribbon-shaped particles or other filamentous microparticles of regular or irregular cross-section, at least the thickness will fall within this range, and so forth. In some embodiments, at least two major dimensions of the microparticle particle fall within this range of dimensions (e.g., at least the thickness and width for an elongated plate, at least the thickness and width for filamentous particles of rectangular, oval, or other regular or irregular cross-section, and so forth). In still other embodiments all major dimensions of the microparticle particle fall within this range of dimensions (e.g., the length, thickness and width of an elongated plate, the length and diameter of a tubular or cylindrical filamentous particle, the length, thickness and width for other filamentous particles of regular or irregular cross-section, etc.).

In certain embodiments, the elongated particles are nanoparticles, by which is meant that at least one major dimension of the particle (e.g., selected from diameter and length for an elongated particle of circular geometry such as a cylindrical or tubular particle, selected from length, width and thickness for an elongated plate or ribbon, and so forth) is less than 100 nm, for example, ranging from 100 nm to 30 nm to 10 nm to 3 nm to 1 nm or less.

In certain further embodiments, the elongated particles are in the form of nanofilaments, by which is meant a filamentous particle in which all cross sectional dimensions taken perpendicular to the major axis along the length of the filament (e.g., the diameter of a tubular or cylindrical filamentous particle, the thickness and width for other filamentous particles of regular or irregular cross-section, etc.) are less than 100 nm, for example, ranging from 100 nm to 30 nm to 10 nm to 3 nm to 1 nm or less. The length of the nanofilament may exceed these dimensions.

In certain embodiments, the filamentous particles are employed which are high aspect ratio particles, by which is meant that the length divided by the greatest cross sectional dimension taken perpendicular to the axis that corresponds to the length of the filamentous particle (e.g., the diameter for a cylindrical or tubular filament, width for a ribbon shaped filament, and so forth) is greater than 10, for example ranging from 10 to 25 to 50 to 100 to 250 to 500 to 1000 or more.

Elongated particles for use in the present invention inherently possess dipoles (sometimes referred to as “permanent dipoles”), or dipoles can be induced in the particles by application of an electric field, or both. As indicated in the background section above, elongated particles having dipoles are known to align themselves in accordance with an applied electric field.

Elongated particles for use in the present invention may be formed from a variety of inorganic and organic materials. Organic materials for the formation of elongated particles may be selected, for instance, from suitable members of the organic materials listed above for use as matrix materials, among others. Such materials may be, for example, materials within which dipoles may be induced and/or materials having a permanent dipole. Examples of the former include conductive polymers. See, e.g., J. Wojturski et al, “Electrical Conductivity of Polyaniline Suspensions 2. Freezing-Melting Cycle,” Croatica Chemica Acta 71 (4) 873-882 (1998). Examples of the latter include nanoparticles in the form of polymer molecules, which have one or more anionic end groups at one end and one or more cationic groups at the other end.

Inorganic materials may likewise be selected, for example, from suitable ceramic materials listed above for use as matrix materials among others. Inorganic materials may also be selected, for example, from suitable metallic materials selected from the following: substantially pure metals (e.g., biostable metals such as gold, platinum, palladium, iridium, osmium, rhodium, titanium, tantalum, tungsten, and ruthenium, and bioresorbable metals such as magnesium and iron), biostable metal alloys such as alloys comprising iron and chromium (e.g., stainless steels, including platinum-enriched radiopaque stainless steel), alloys comprising nickel and titanium (e.g., nitinol), alloys comprising cobalt and chromium, including alloys that comprise cobalt, chromium and iron (e.g., elgiloy alloys), alloys comprising nickel, cobalt and chromium (e.g., MP 35N) and alloys comprising cobalt, chromium, tungsten and nickel (e.g., L605), alloys comprising nickel and chromium (e.g., inconel alloys), and bioabsorbable metal alloys such as magnesium alloys and iron alloys (including their combinations with Ce, Ca, Zn, Zr and Li), among many others.

Additional examples of elongated particles, not necessarily exclusive of those above, may be selected from suitable members of the following: carbon nanotubes, carbon fibers, magnetite nanowires, alumina fibers, titanium oxide fibers, tungsten oxide fibers, silica fibers, tantalum oxide fibers, zirconium oxide fibers, silicate fibers such as aluminum silicate nanofibers and attapulgite clay, and synthetic or natural phyllosilicates including clays and micas such as montmorillonite, hectorite, hydrotalcite, vermiculite and laponite, among many others.

Specific examples of carbon nanotubes include single wall carbon nanotubes (SWNTs), which typically have outer diameters ranging from 0.25 nanometer to 5 nanometers, and lengths up to 10's of micrometers or more, and multi-wall carbon nanotubes (including so-called “few-wall” nanotubes), which typically have inner diameters ranging from 2.5 nanometers to 10 nanometers, outer diameters of 5 nanometers to 50 nanometers, and lengths up to 10's of micrometers or more, among others.

The elongated particles for use in the present invention, including various organic and inorganic (e.g., carbon, metallic, ceramic, etc.) particles, may be derivatized with a variety of chemical entities. For example the particles may be covalently linked or “functionalized” with the chemical entities, or they may be otherwise associated with the chemical entities (e.g., by non-covalent interactions, encapsulation, etc.). Derivatization may result, for example, in improved processing, improved compatibility with the surrounding matrix material, and so forth. Although the discussion that follows is largely directed to techniques for derivatizing carbon particles, such as carbon nanotubes and nanofibers, analogous and non-analogous methods may also be employed to derivatize other particles.

For example, in some embodiments of the invention, particles are functionalized with simple organic and inorganic groups. For example, the functionalization of carbon particles with carboxyl, amino, halogen (e.g., fluoro), hydroxyl, isocyanate, acyl chloride, amido, ester, and O₃ functional groups has been reported, among others. See, e.g., K. Balasubramanian and M. Burghard, “Chemically Functionalized Carbon Nanotubes,” Small 2005, 1, No. 2, 180-192; T. Ramanathan et al., “Amino-Functionalized Carbon Nanotubes for Binding to Polymers and Biological Systems,” Chem. Mater. 2005, 17, 1290-1295; C. Zhao et al., “Functionalized carbon nanotubes containing isocyanate groups,” Journal of Solid State Chemistry, 177 (2004) 4394-4398; and S. Banerjee et al., “Covalent Surface Chemistry of Single-Walled Carbon Nanotubes,” Adv. Mater. 2007, 17, No. 1, January 6, 17-29. As indicated above, such groups may be provided to improve suspendibility of the particles, to improve interactions with the surrounding matrix, and so forth.

In some embodiments of the invention, elongated particles are functionalized with polymers. For example, polymer functionalized carbon particles have been formed using so-called “grafting to” and “grafting from” approaches.

In the “grafting to” approach, pre-formed polymers are attached to particle surfaces. In a typical procedure, the preformed polymer has one or more reactive groups (e.g., reactive side or end groups) which may be directly reacted with functional groups on the particles or which are linked to functional groups on the particles by intermediate coupling species. An advantage of the “grating to” approach is that it allows for the complete characterization and control of the polymers prior to grafting them to the particles.

As a specific example, carboxyl- and acyl-chloride-functionalized carbon nanotubes may be conjugated to hydroxyl- and amino-terminated polymers, via ester and amide linkages, respectively, to form polymer-functionalized nanotubes. For instance, carbon nanotubes functionalized with carboxyl groups (—COOH) or acyl chloride groups (—CO—Cl) have been reacted with hydroxyl terminated polymers such as hydroxyl terminated polyethylene glycol and hydroxyl terminated polystyrene. See, e.g., C. Baskaran et al., “Polymer adsorption in the grafting reactions of hydroxyl terminal polymers with multi-walled carbon nanotubes,” Polymer 46 (2005) 5050-5057. Also, Menna et al., “Shortened single-walled nanotubes functionalized with poly(ethylene glycol): preparation and properties,” ARKAT 2003 (xiii) 64-73, describe reaction of amino-terminated poly(ethylene glycol), with acid chloride functionalized carbon nanotubes. In R. Czerw et al., “Organization of Polymers onto Carbon Nanotubes: A Route to Nanoscale Assembly,” Nano Lett., Vol. 1, No. 8, 2001, 423-427, acyl chloride functionalized nanotubes are reacted with poly-(propionylethylenimine-co-ethylenimine) (PPEI-EI) thereby attaching the PPEI-EI to the nanotubes via amidation. Also described is the attachment of poly(vinyl acetate-co-vinyl alcohol) to acyl chloride functionalized nanotubes via ester linkages.

As another specific example, carbon nanotubes functionalized with amino groups have been reported to make possible bonding to a variety of synthetic and organic polymers, including poly(methyl methacrylate), poly(acrylic acid), DNA and carbohydrates. See T. Ramanathan et al., “Amino-Functionalized Carbon Nanotubes for Binding to Polymers and Biological Systems,” Chem. Mater. 2005, 17, 1290-1295.

As another specific example, N-protected amino acids have been linked to carbon nanotubes and subsequently used to attach peptides via fragment condensation or using a maleimido linker. See, e.g., S. Banerjee et al., “Covalent Surface Chemistry of Single-Walled Carbon Nanotubes,” Adv. Mater. 2007, 17, No. 1, January 6, 17-29.

As yet another specific example, radical coupling between polymer chain ends and single wall nanotubes has been reported. In this method, nitroxide-mediated polymerization is used to produce well-defined polymers, in this instance, polystyrene and poly[(tert-butyl acrylate)-b-styrene], with nitroxide end groups. By heating these nitroxide terminated polymers, chain-end radicals are produced that undergo coupling to single-walled carbon nanotubes through a radical coupling reaction. This allows for the functionalization of single-walled carbon nanotubes with well-defined polymers, including polystyrene and poly[(tert-butyl acrylate)-b-styrene], among others. The tert-butyl groups of the appended poly[(tert-butyl acrylate)-b-styrene] may be removed to produce poly[(acrylic acid)-b-styrene]-functionalized carbon nanotubes. For further information, see Liu, Y. et al, “Functionalization of Single-Walled Carbon Nanotubes with Well-Defined Polymers by Radical Coupling,” Macromolecules, 2005, 38, 1172-1179.

C. Zhao et al., “Functionalized carbon nanotubes containing isocyanate groups,” Journal of Solid State Chemistry, 177 (2004) 4394-4398, describe formation of functionalized carbon nanotubes that contain aromatic isocyanate groups, specifically toluene 2-isocyanate groups. Isocyanates are reactive, with reactions commonly occuring through addition to C═N double bond. Aromatic isocyanates are generally more reactive than aliphatic ones. Isocyanates react quite readily with amines, including primary aliphatic amines (R—NH₂), primary aromatic amines (Ar—NH₂) and secondary aliphatic amines (RR′NH), and these reactions are commonly conducted without catalysis (R, R′, etc. are aliphatic groups, Ar is an aromatic group). Isocyanate reactivity with alcohols, including those having primary (RCH₂—OH), secondary (RR′CH—OH) and tertiary (RR′R″C—OH) hydroxyls, is moderate and may be catalyzed by bases, such as tertiary amines or organometals. Isocyanates also react with carboxylic acids (RCOOH), ureas (R—NH—CO—NH—R), urethanes (RR′R″C—OH), and amides (RCO—NH₂). Thus, polymers having these groups (e.g., as end groups), may be coupled to isocyanate functionalized carbon nanotubes such as those described in C. Zhao et al.

Fluorine atoms in fluorinated carbon nanotubes may be replaced through nucleophilic substitution reactions, for example, with alcohols, amines, Grignard reagents, and alkyl lithium compounds. See K. Balasubramanian and M. Burghard, “Chemically Functionalized Carbon Nanotubes,” Small 2005, 1, No. 2, 180-192. Hence, polymers with hydroxyl (e.g., a polymer comprising a —CH₂—OH moiety, etc.), amino (e.g., a polymer comprising a —CH₂—NH₂ moiety, etc.), alkyllithium (e.g., a polymer comprising a —CH₂—Li moiety, etc.) or Grignard (e.g., a polymer comprising a —CH₂—MgBr moiety, etc.) may be grafted to fluorinated carbon nanotubes via a nucleophilic substitution.

Based on the forgoing, suitable linking chemistries may be selected from following, among others: (a) linking chemistries in which polymers containing amino groups (e.g., amino terminated polymers, among others) are linked to carboxyl-, acyl-chloride-, isocyanate- or fluorine-functionalized particles; (b) linking chemistries in which polymers containing hydroxyl groups (e.g., hydroxyl terminated polymers among others) are linked to carboxyl-, acyl chloride-, isocyanate-, or fluorine-functionalized particles, among others; (c) linking chemistries in which polymers containing carboxyl groups (e.g., carboxyl terminated polymers, among others) are linked to amino- and isocyanate-functionalized particles, and (d) linking chemistries in which polymers containing Grignard or alkyllithium groups (e.g., Grignard or alkyllithium terminated polymers, among others) are linked to halogen-functionalized particles.

Turning now to “grafting from” approaches, polymerization typically proceeds in these methods from an initiation site at the surface of the particle. “Grafting from” techniques typically involve (a) the attachment of polymerization initiators to the particles surfaces, followed by (b) polymerization of monomers from the resulting particle-based macroinitiator.

A variety of polymerization techniques may be employed in “grafting from” techniques, including so-called “living” cationic, anionic and radical polymerization techniques, examples of which include atom transfer radical polymerization (ATRP), stable free-radical polymerization (SFRP), nitroxide-mediated processes (NMP), and degenerative transfer (e.g., reversible addition-fragmentation chain transfer (RAFT)) processes, among others. The advantages of using a “living” free radical method for polymer synthesis include non-stringent reaction conditions, molecular weight control, and the ability to prepare block copolymers by the sequential activation of a dormant chain end in the presence of different monomers. These methods are well-detailed in the literature and are described, for example, in an article by Pyun and Matyjaszewski, “Synthesis of Nanocomposite Organic/Inorganic Hybrid Materials Using Controlled/“Living” Radical Polymerization,” Chem. Mater., 13:3436-3448 (2001).

ATRP is a particularly popular free radical polymerization technique, as it is tolerant of a variety of functional groups (e.g., alcohol, amine, carboxylic, acid, sulfonate, etc. groups). In polymerizations of monomers via ATRP, radicals are commonly generated by the redox reaction of organic halide initiators such as alkyl halides with transition-metal complexes. Some typical examples of organic halide initiators include haloesters (e.g., methyl 2-bromopropionate, ethyl 2-bromoisobutyrate, etc.) and benzyl halides (e.g., 1-phenylethyl bromide, benzyl bromide, etc.). A wide range of transition-metal complexes may be employed, including a variety of Ru—, Cu—, and Fe-based systems. Examples of monomers that may be used in ATRP polymerization reactions include various unsaturated monomers such as alkyl methacrylates, alkyl acrylates, hydroxyalkyl methacrylates, vinyl esters, and vinyl aromatic monomers, among others.

A general strategy for grafting polymers from carbon nanotubes via ATRP is set forth in H. Kong et al., “Controlled Functionalization of Multiwalled Carbon Nanotubes by in Situ Atom Transfer Radical Polymerization,” J. Am. Chem. Soc., Vol. 126, No. 2, 2004, 412-413. This general strategy includes the following steps: (1) nanotubes functionalized with carbonyl chloride groups (also referred to herein as acyl chloride groups) are prepared via reaction of thionyl chloride with carboxyl-containing nanotubes previously made by the oxidation of the nanotubes with 60% HNO₃, (2) the carbonyl chloride functionalized nanotubes are reacted with ethylene glycol, generating hydroxyl-functionalized nanotubes, (3) initiating sites for ATRP are formed by reacting the hydroxyl functionalized nanotubes with 2-bromo-2-methylpropionyl bromide, and (4) polymerization from the 2-bromo-2-methylpropionate functionalized nanotubes is carried out by means of ATRP. Nanotubes functionalized with poly(methyl methacrylate) chains are specifically described. The thickness of the polymer layer (i.e., chain length) is controlled by the varying the ratio of the methyl methacrylate to the 2-bromo-2-methylpropionate functionalized nanotubes.

In Z. Yao et al., “Polymerization from the Surface of Single-Walled Carbon Nanotubes-Preparation and Characterization of Nanocomposites,” J. Am. Chem. Soc., 2003, 125, 16015-16024, single-walled carbon nanotubes are functionalized with phenol groups using the 1,3-dipolar cycloaddition reaction. These phenols are further derivatized with 2-bromoisobutyryl bromide, yielding nanotubes with attached atom transfer radical polymerization initiators. These initiators are active, with the polymerization of methyl methacrylate and tert-butyl acrylate from the surfaces of the nanotubes being reported.

Similarly, polystyrene has been grown from single wall nanotubes by ATRP, which is initiated with 2-bromopropionate groups immobilized on single wall nanotubes. The nanotube initiator, 2,2′-bipyridine, and styrene monomer are combined in 1,2-dichlorobenzene, and polymerization is performed at 110° C. in the presence of CuBr. Methyl 2-bromopropionate may be added as a free initiator to control the chain propagation from the solid surface and to monitor the polymerization kinetics. For further details, see S. Qin et al., “Functionalization of Single-Walled Carbon Nanotubes with Polystyrene via Grafting to and Grafting from Methods,” Macromolecules 2004, 37, 752-757.

Beyond ATRP, the polymerization of norbornene from Grubbs catalyst-functionalized carbon nanotubes has also been demonstrated using ring-opening metathesis polymerization (ROMP). For more information see Y. Liu and A. Adronov, “Preparation and Utilization of Catalyst-Functionalized Single-Walled Carbon Nanotubes for Ring-Opening Metathesis Polymerization,” Macromolecules, 2004, 37, 4755-4760.

Anionic polymerization from carbon nanotubes has been reported in G. Viswanathan, “Single-Step in Situ Synthesis of Polymer-Grafted Single-Wall Nanotube Composites,” J. Am. Chem. Soc., Vol. 125, No. 31, 2003, 9258-9. In this reference, carbon nanotubes are dispersed in purified cyclohexane, after which sec-butyllithium is added to the dispersion in slight excess, to ensure the removal of protic impurities on the nanotube surfaces. According to the authors, carbanions are introduced on the nanotube surfaces, thereby providing initiating sites for the polymerization of styrene. Styrene monomer is then added and polymerized to form polystyrene-functionalized nanotubes.

Thus, using “grafting from” techniques such as those based on anionic, cationic and free radical “living” polymerization techniques, a variety of homopolymers and copolymers may be grown from particle surfaces.

Moreover, as previously discussed, a wide variety of homopolymers and copolymers may be formed and subsequently attached to the particle surfaces using suitable “grafting to” techniques.

Further information specifically related to polymer derivation of carbon nanotubes may be found, for example, in C. Wang et al., “Polymers containing fullerene or carbon nanotube structures, Prog. Polym.Sci. 29(2004) 1079-1141.

Examples of homopolymers and copolymers which may be attached to (e.g., “grafted to” or “grafted from”) particles for use in the present invention, include suitable polymers set forth above for use as matrix materials, among others.

In some embodiments, the attached homopoloymers and copolymers are selected to match, as closely as is practical, the properties of the matrix material.

For instance, polyether-block-polyamides are described as examples of matrix materials, in which case it may be desirable to derivatize the elongated particles with polyethers, polyamides, or polyether-block-polyamides. Numerous examples of these polymers are described above. Specific examples of polyethers include polyether homopolymers and copolymers such as those containing one or more of the following: ethylene oxide, trimethylene oxide, propylene oxide and polytetramethylene oxide, among others. Specific examples of polyamides include polyamide homopolymers and copolymers such as nylon 6, nylon 4/6, nylon 6/6, nylon 6/10, nylon 6/12, nylon 11 and nylon 12, among others.

Similarly, poly(vinyl aromatics) are described as examples of matrix materials, in which case it may be desirable to derivatize the elongated particles with polyalkenes, poly(vinyl aromatics), or polyalkenes-block-poly(vinyl aromatics). Numerous examples of these polymers are described above. Specific examples of polyalkenes include polyalkene homopolymers and copolymers such as those containing one or more of the following: ethylene, butylene and isobutylene, among others. Specific examples of poly(vinyl aromatics) include poly(vinyl aromatic) homopolymers and copolymers such as those containing one or more of the following: styrene and alpha-methyl-styrene, among others.

As another specific example, ceramic materials such those comprising alumina, zirconia, glass-ceramics, calcium phosphate, or a combination thereof, among others, may be used herein as matrix materials, in which it may be desirable to derivatize the elongated particles with hydrophilic polymers, for example, polyethers. Specific examples of polyethers include polyether homopolymers and copolymers such as those containing one or more of the following: ethylene oxide, trimethylene oxide, propylene oxide and polytetramethylene oxide, among others.

In some aspects of the invention, the particles are derivatized with polyoxometallates (POMs).

POMs are a large class of nanosized, anionic, metal and oxygen containing molecules. Polyoxometalates have been synthesized for many years (the first known synthesis dates back to 1826), they readily self assemble under appropriate conditions (e.g., acidic aqueous media), and they are quite stable. POMs comprise one or more types of metal atoms, sometimes referred to as addenda atoms (commonly molybdenum, tungsten, vanadium, niobium, tantalum or a mixture of two or more of these atoms), which with the oxygen atoms form a framework (sometimes referred to as the “shell” or “cage”) for the molecule. More specific examples include V^{V}, Nb^{V}, Mo^{VI} and W^{VI}, among others. Some POMs further comprise one or more types of central atoms, sometimes referred to as heteroatoms, which lie within the shell that is formed by the oxygen and addenda atoms. A very wide variety of elements (i.e., a majority of elements in the periodic table) may act as heteroatoms, with some typical examples being P⁵⁺, As⁵⁺, Si⁴⁺, Ge⁴⁺, B³⁺, and so forth. In certain cases, one or more of the oxygen atoms within the POM is/are substituted by S, F, Br and/or other p-block elements. Materials for forming POMs may be obtained, for example, from Sigma Aldrich and Goodfellow Corp., among other sources.

Derivatized POMs are being developed constantly in which organic compounds, including polymers and non-polymers, are covalently linked or otherwise associated with POMs. Examples include POM derivatives where one or more organic compounds are covalently bonded directly to the POM framework (e.g., to addenda atoms) and/or bonded to POM heteroatoms. For instance, POM derivatives may be prepared by a variety of techniques, including techniques where organic compounds are covalent bound to POM addenda atoms or heteroatoms by imido linkages. For further information, see, e.g., Peng, Z., “Rational synthesis of covalently bonded organic-inorganic hybrids,” Angew Chem Int Ed Engl. Feb. 13, 2004; 43(8), 930-5; Moore, A. R. et al., “Organoimido-polyoxometalates as polymer pendants,” Chem. Commun. 2000, 1793-1794; Hu Changwen et al., “Polyoxometalate-based organic-inorganic hybrid materials,” C.J.I. Jun. 1, 2001 3(6), 22; P. Wu et al., “An Easy Route to Monofunctionalized Organoimido Derivatives of the Lindqvist Hexmolybdate,” Eur. J. Inorg. Chem. 2004, 2819-2822; M. Lu et al. “Synthesis of Main-Chain Polyoxometalate-Containing Hybrid polymers and Their Applications in Photovoltaic Cells,” Chem. Mater. 2005, 17, 402-408. J. Zhang et al., “Improving multilayer films endurance by photoinduced interaction between Dawson-type polyoxometalate and diazo resion,” Materials chemistry and Physics 90 (2005) 47-52. POMs such as organoimido POM derivatives, which have strong d-π interaction between the organic delocalized π electrons and the cluster d electrons, are preferred in certain embodiments. See, e.g., C. Qin et al., “A linear bifunctionalized organoimido derivative of hexamolybdate: Convenient synthesis and crystal structure,” Inorganic Chemistry Communications 8 (2005) 751-754, and references cited therein.

For example, permanent dipole entities suitable for electrical alignment may be created by coupling a monofunctionalized polyoxometalate (as noted above, polyoxometalates are negatively charged) to a positively charged organic compound, such as a positively charged polymer (e.g., via a reactive end-group on the positively charged polymer) or a positively charged non-polymer. Examples of positively charged polymers may be selected from suitable positively charged polymers set forth above for use as matrix materials, and from suitable polycations listed below for use in layer-by-layer techniques. Charged polymers may also be polymerized in a “grafting from” type procedure, using polyoxometalates with suitable initiators attached.

Moreover, functionalized polyoxometalates may be coupled to positively charged particles (e.g., amine-functionalized particles such as the amine-functionalized carbon nanotubes described above, among others), thereby establishing a permanent dipole. Examples of positively charged ceramic nanoparticles include titanium oxide nanoparticles or ruthenium nanoparticles such as those described, for example, in Jun Yang et al., “Preparation and characterization of positively charged ruthenium nanoparticles,” Journal of Colloid and Interface Science 271 (2004) 308-312).

Furthermore, polyoxometalates may be coupled to particles within which a dipole may be induced upon being subjected to an electric field (e.g., a carbon nanotube, among others).

For example, a polyoxometalate having one or more covalently attached organic compounds, including attached polymeric and non-polymeric moieties (e.g., organoimido derivatives such as the organoimido derivatives of C. Qin, Inorganic Chemistry Communications 8 (2005) 751-754 or the halogenated arylimido polyoxometalate derivatives described in P. Wu et al., Eur. J Inorg. Chem. 2004, 2819-2822 or ido- or ethynyl-functionalized monomeric and polymeric polyoxometalates such as those described in M. Lu et al., Chem. Mater. 2005, 17, 402-408, among others) may be covalently linked to other species including, for example, functionalized carbon nanotubes, either directly or through a polymer or non-polymer coupling agent. For example, a polymer chain with two functional groups may be employed as a coupling agent: one to attach to the polyoxometalate and the other to attach the carbon nanotube.

As another example, carbon nanotubes may be functionalized with isocyanate groups or amine groups as described, for example, in C. Zhao et al., “Functionalized carbon nanotubes containing isocyanate groups,” Journal of Solid State Chemistry, 177 (2004) 4394-4398 and Ramanathan et al., “Amino-Functionalized Carbon Nanotubes for Binding to Polymers and Biological Systems,” Chem. Mater. 2005, 17, 1290-1295, respectively. Subsequently, polyoxometalate-nanotube hybrids may be formed via reactions between the polyoxometalates and the isocyanate or amine groups on the nanotubes as described, for example, in R. A. Roesner et al., “Mono- and di-functional aromatic amines with p-alkoxy substituents as novel arylimido ligands for the hexamolybdate ion,” Inorganica Chimica Acta 342 (2003) 37-47.

Elongated particles containing polyoxometalates may be employed, for example, where the matrix material is at least partially hydrophilic. For example, such particles may be used where the matrix material is a polyether or a polyether-block-polyamide such as those described above, among others. Alternatively, they may be used where the matrix material is a ceramic material such as one comprising alumina, zirconia, glass-ceramics, calcium phosphate, or a combination thereof, among others.

D. Formation of Particle-Containing Regions.

Typically, methods of forming particle-containing regions in accordance with the present involve subjecting a liquid suspension of the elongated particles to an electrical field to align them. Once the elongated particles are aligned, the liquid suspension may be solidified, if necessary, to fix the elongated particles in their new orientation.

Examples of suspensions meeting these criteria include particle suspensions within polymer melts (e.g., where polymers having thermoplastic characteristics are employed as matrix materials), within polymer solutions (e.g., where the polymers that are employed as matrix materials are dissolvable in an aqueous or organic solvent), within curable polymer systems (e.g., systems such as epoxy systems which undergo chemical cure, and systems that cure upon exposure to radiation, including UV light and heat), and within liquid suspensions that further include ceramic particles, among others.

Examples of polymer processing techniques include those techniques in which a solution (e.g., where solvent-based processing is employed), melt (e.g., where thermoplastic processing is employed), or other liquid polymer composition (e.g., where a curable composition is employed) containing elongated particles is applied to a substrate. For example, the substrate can correspond to all or a portion of a medical article surface to which a layer is applied. The substrate can also be, for example, a template, such as a mold, from which the particle-containing region is separated after formation. In other embodiments, for example, extrusion and co-extrusion techniques, particle-containing regions may be formed without the aid of a substrate. In all cases an electric field is applied to align the elongated particles prior to immobilization of the same, for example, due to solidification of the polymer (e.g., as a result of cooling, solvent evaporation, cross-linking, etc.)

Specific examples of polymer processes include molding, casting and coating techniques such as injection molding, blow molding, solvent casting, dip coating, spin coating, spray coating, coating with an applicator (e.g., by roller or brush), web coating, screen printing, and ink jet printing, as well as extrusion into sheets, fibers, rods, tubes and other cross-sectional profiles of various lengths.

Particle-containing regions in accordance with the present invention may also be created from a liquid suspension of elongated particles by processes commonly known as layer-by-layer techniques, by which a variety of substrates may be coated using charged materials via electrostatic self-assembly. In the layer-by-layer technique, a first layer having a first surface charge is typically deposited on an underlying substrate (e.g., a medical device or portion thereof, a template, such as a mold, from which the particle-containing regions is separated after formation, etc.), followed by a second layer having a second surface charge that is opposite in sign to the surface charge of the first layer, and so forth. The charge on the outer layer is reversed upon deposition of each sequential layer. Commonly, 5 to 10 to 25 to 50 to 100 to 200 or more layers are applied in this technique, depending on the desired thickness.

Layer-by-layer techniques generally employ charged polymer species, including those commonly referred to as polyelectrolytes. Specific examples of polyelectrolyte cations (also known as polycations) include protamine sulfate polycations, poly(allylamine) polycations (e.g., poly(allylamine hydrochloride) (PAH)), polydiallyldimethylammonium polycations, polyethyleneimine polycations, chitosan polycations, gelatin polycations, spermidine polycations and albumin polycations, among many others. Specific examples of polyelectrolyte anions (also known as polyanions) include poly(styrenesulfonate) polyanions (e.g., poly(sodium styrene sulfonate) (PSS)), polyacrylic acid polyanions, sodium alginate polyanions, eudragit polyanions, gelatin polyanions, hyaluronic acid polyanions, carrageenan polyanions, chondroitin sulfate polyanions, and carboxymethylcellulose polyanions, among many others.

The layer-by-layer techniques will also employ a polarized or polarizable elongated particle which also has an overall negative or positive charge. As a specific example, a suspension of negatively charged carbon nanotubes (with or without an accompanying anionic polyelectrolyte) may be employed for the deposition of one or more negatively charged layers. The elongated particles may be aligned during the deposition process by applying an electric field as discussed below.

As previously discussed, in addition to organic materials such as polymers, matrix materials in accordance with the present invention also include inorganic materials, such as ceramic materials. Ceramic processing may proceed by a variety of techniques, such as those in which liquid suspensions of ceramic particles are processed (e.g., colloid based processing). Suitable examples of ceramic processing techniques based on liquid suspensions may be selected, for example, from coating techniques such as dip-coating, spray coating, coating with an applicator (e.g., by roller or brush), spin-coating, ink-jet printing or screen printing, as well as various casting/molding techniques, including slip casting, tape casting, direct coagulation casting, electrophoretic casting, gelcasting, hydrolysis assisted solidification, aqueous injection molding, and temperature induced forming. Analogous to the above techniques, elongated particles may be provided within the liquid suspensions and aligned using an electric field prior to solidification of the suspensions. In this way, these techniques may be used to form particle-containing regions, typically in conjunction with a substrate, such as a medical device or portion thereof, or a template such as a mold from which the particle-containing regions is separated after formation.

Sol-gel processing will now be described in more detail, with the understanding that other ceramic processing techniques, including other techniques based on liquid suspensions of solid ceramic particles, may be employed. In a typical sol-gel process, precursor materials, typically selected from inorganic metallic and semi-metallic salts, metallic and semi-metallic complexes/chelates, metallic and semi-metallic hydroxides, and organometallic and organo-semi-metallic compounds such as metal alkoxides and alkoxysilanes, are subjected to hydrolysis and condensation (also referred to sometimes as polymerization) reactions, thereby forming a “sol” (i.e., a suspension of solid particles within a liquid).

For example, an alkoxide of choice (such as a methoxide, ethoxide, isopropoxide, tert-butoxide, etc.) of a semi-metal or metal of choice (such as silicon, aluminum, zirconium, titanium, tin, hafnium, tantalum, molybdenum, tungsten, rhenium, iridium, etc.) may be dissolved in a suitable solvent, for example, in one or more alcohols. Subsequently, water or another aqueous solution, such as an acidic or basic aqueous solution (which aqueous solution can further contain organic solvent species such as alcohols) is added, causing hydrolysis and condensation to occur. If desired, additional agents can be added, such as agents to control the viscosity and/or surface tension of the sol. Moreover, elongated particles are also provided within the sol, in accordance with the invention.

Further processing of the sol enables solid materials to be made in a variety of different forms. For instance, coatings can be produced on a substrate by spray coating, coating with an applicator (e.g., by roller or brush), spin-coating, dip-coating, ink-jet printing, screen printing, and so forth, of the sol onto the substrate, whereby a “wet gel” is formed. Monolithic wet gels can be formed, for example, by placing the sol into or onto a mold or another form (e.g., a sheet). Elongated particles within the wet gel may be aligned as discussed elsewhere herein during the wet gel stage. The wet gel is then dried. Further information concerning sol-gel materials can be found, for example, in Viitala R. et al., “Surface properties of in vitro bioactive and non-bioactive sol-gel derived materials,” Biomaterials, August 2002; 23(15):3073-86.

For particle alignment, either AC or DC electrical fields may be used. Both have been employed in epoxy composites containing carbon nanotubes or and carbon nanofibers. See, e.g., T. Prasse et al., “Electric anisotropy of carbon nanofibre/epoxy resin composites due to electric field induced alignment,” Composites Science and Technology 63 (2003) 1835-1841; and C. A. Martin et al., “Electric field-induced aligned multi-wall carbon nanotube networks in epoxy composites,” Polymer 46 (2005) 877-886. Fields of 50 to 800 V/cm and frequencies of 50 Hz to 10 kHz were employed.

Applying liquid suspensions of elongated particles to a substrate in multiple layers allows one to change the direction of the particles from layer to layer. For example, one may change the direction of the electric field between layers such that the particles within alternating layers are aligned perpendicular to each other (e.g., for a device of circular cross section, one layer may be circumferentially aligned and another axially aligned, or one layer one layer may be circumferentially aligned and another radially aligned, and so forth). One could, of course, employ a single preferential direction for a single layer or for multiple layers.

Where the elongated particles have a net charge, it may be desirable to employ an AC electric field to minimize or eliminate migration of the particles within the suspension (e.g., to prevent electrode agglomeration of the particles). On the other hand, it may be desirable to promote gradients in elongated particle density, as well as particle alignment, in which case DC electric fields, or combinations of DC and AC electric fields (e.g., by applying an alternating voltage, which a DC bias), may be employed.

A few examples of electrode arrangements which may be employed, among many other possibilities, will now be described.

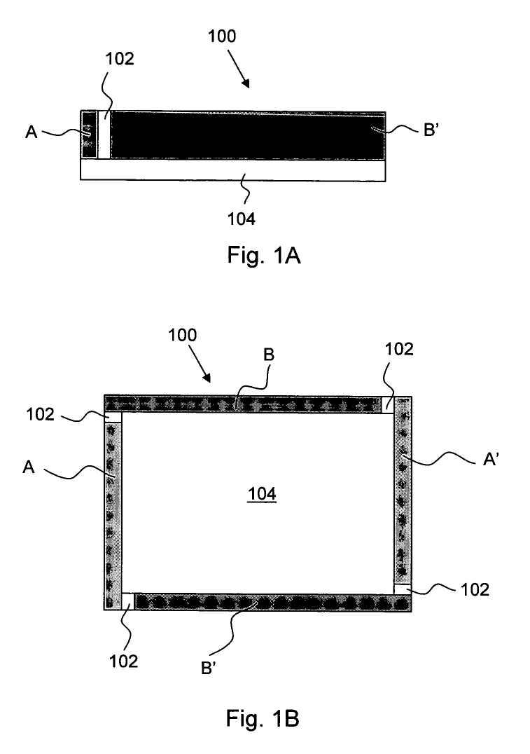

FIGS. 1A and 1B are side and top views, respectively, of an apparatus 100 in which elongated particles may be aligned, in accordance with the present invention. The apparatus includes sides that are formed from conductive electrodes A, A′, B and B′ and insulating portions 102, which electrically insulate the electrodes A, A′, B and B′ from one another. The apparatus also includes a bottom 104, which may correspond to a medical device or portion thereof, or which may correspond to a template from which the particle-containing region that is formed may subsequently be removed.

The apparatus 100 contains two sets of electrodes A,A′ and B,B′ that are positioned to contact a liquid suspension of elongated particles, which may be selected, for example, from those discussed above, among others. With reference to FIG. 1B, elongated particles may be aligned horizontally relative to the page by a applying a suitable voltage across electrodes A-A′ and may be aligned vertically relative to the page by a applying a suitable voltage across electrodes B-B′. The liquid suspension may then be solidified, if necessary, to set the particles in the alignment that is generated by the applied voltage. In general, the electric field will be applied during at least a portion of the solidification process.

In certain embodiments, a first solidified layer is prepared, in which the elongated particles are aligned in a first orientation, after which a second solidified layer is prepared, in which the elongated particles are aligned in a second orientation that differs from the first orientation. Additional layers may be created as desired.

In this manner, using an apparatus like apparatus 100 of FIGS. 1A and 1B, alternating layers may be created which contain elongated particles that are aligned perpendicularly to one another. For instance, during formation of the first, third, fifth, etc. layers, one may apply an AC field between electrodes A and A′, whereas during formation of the second, fourth, sixth, etc. layers, one may apply an AC field between electrodes B and B′. Of course, many other combinations of angles and layer configurations are possible.

The apparatus of FIGS. 1A and 1B is useful for aligning elongated particles within planar regions. Examples of medical devices within with such particle-containing regions may be employed include heart valves, orthopedic plates, intraocular contact lenses, leaves to be used in venous valves, and so forth.

Of course, planar regions may then be bent into a tubular configuration after formation, or they may be otherwise bent or folded, depending upon the ultimate application.

In the case where a particle-containing region in accordance with the present invention is formed on a cylindrical or tubular substrate (e.g., on a cylindrical or tubular mold, or on a cylindrical or tubular medical device structure such as a stent or a balloon), particle alignment along the axis of the device is relatively simple, because all of the particles are oriented in the same direction.

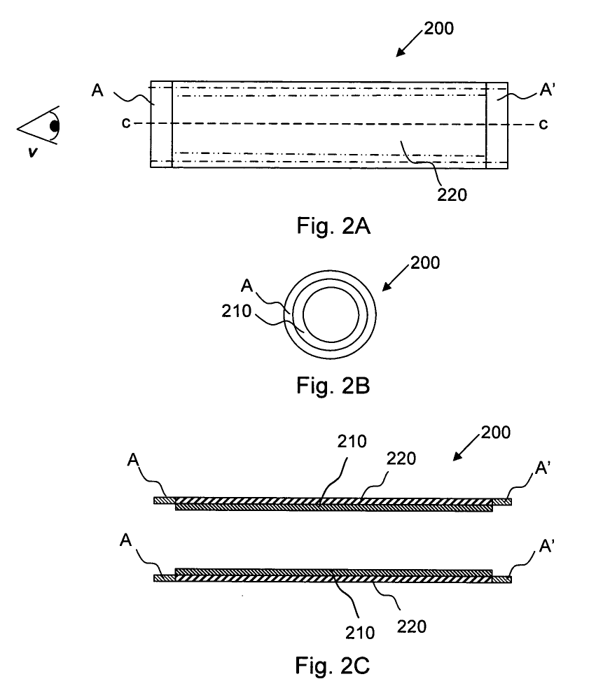

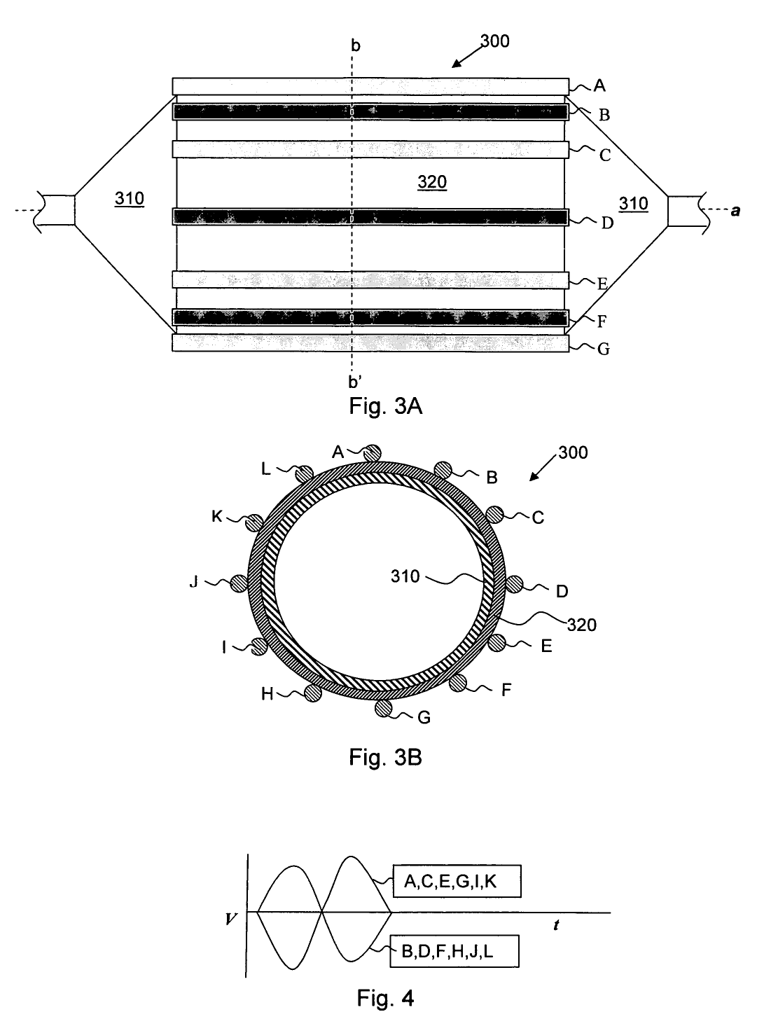

For example, an apparatus 200 is shown in FIGS. 2A-C with which elongated particles may be aligned along the length of a medical device. FIG. 2A is a side view of the apparatus, whereas FIG. 2B is an end view taken along view v of FIG. 2A and FIG. 2C is a cross sectional view of the device taken along the plane corresponding to the line c-c of FIG. 2A. In these figures, a stent 210 is shown, to whose outer surface has been applied a liquid suspension of elongated particles 220, for example, using a technique selected from those discussed above, among others. The elongated particles within the suspension may be aligned along the length of the device by applying a suitable voltage across ring shaped electrodes A and A′. The liquid suspension may then be solidified, if necessary, to set the particles in the alignment that is generated by the applied voltage. A gradient in the density of the particles in the radial direction may be obtained by spinning the device 220 around the axis while electrically aligning the particles in axial direction at the same time.

Where the elongated particles are to be aligned around the circumference of a tubular medical device, the situation is more complex. A side view of one example of an apparatus 300 for alignment of elongated particles around the circumference of a cylindrical or tubular substrate (e.g., a cylindrical or tubular mold or a cylindrical or tubular medical device), is illustrated in FIG. 3A. A cross-sectional view of the apparatus of FIG. 3A, taken along the plane corresponding to line b-b, is illustrated in FIG. 3B.

Referring now to these figures, there is shown a tubular substrate, specifically a tubular medical device such as a balloon 310, to whose outer surface has been applied a liquid suspension of elongated particles 320, for example, using a technique selected from those previously discussed, among others. The elongated particles within the suspension 320 may be oriented around the circumference of the device by applying a suitable voltage scheme to electrodes A, B, C, D, E, F, G, H, I, J, K, L, which run parallel to the longitudinal axis a of the balloon 320 and which are spaced approximately equally from one another around the circumference of the balloon. Although twelve electrodes are shown, additional or fewer electrodes may also be employed, with additional electrodes giving finer spatial control.

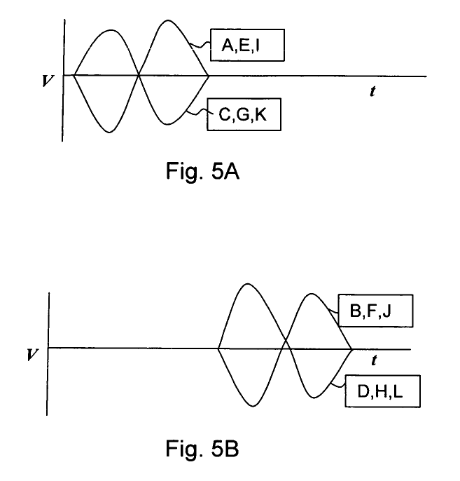

In one scheme, an AC voltage is applied to the electrodes such that the phases of the neighboring electrodes around the circumference of the device have a 180 degree phase shift from one another. Such a scheme is illustrated in FIG. 4, in which the waveform of the voltage V applied to electrodes A, C, E, G, I, K is phase shifted 180 degrees from the waveform of the voltage V applied to electrodes B, D, F, H, J, L over time 1. Although only one complete cycle is illustrated, in practice, more, frequently many more, cycles may be employed to achieve the desired degree of particle alignment.

In order to improve alignment underneath the electrodes, one could rotate the electrodes around the central axis while applying the electric field.

Alternatively, electronic switching may be used during a first time interval to create an electric field between (1) electrodes A and C, (2) electrodes C and E, (3) electrodes E and G, (4) electrodes G and I, (5) electrodes I and K, and (6) electrodes K and A as shown in FIG. 5A, followed by a second time interval in which an electric field is created between (1) electrodes B and D, (2) electrodes D and F, (3) electrodes F and H, (4) electrodes H and J, (5) electrodes J and L and (6) electrodes L and B. Again, only one full cycle is shown in FIGS. 5A and 5B, although many more cycles may be employed. Moreover, the first and second time intervals may be repeated numerous times.

A gradient in the density of the particles in the radial direction may be obtained by spinning the apparatus 300 around its axis while at the same time electrically aligning the particles in a circumferential direction. It will be understood that the electronic switching frequency is much higher then the frequency of rotation.

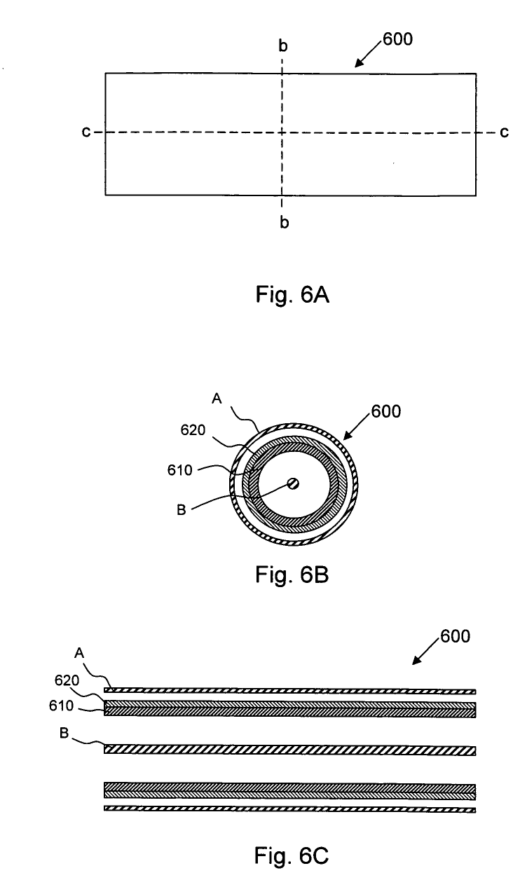

After or during particle alignment, the liquid suspension, may be solidified (e.g., based on one of the mechanisms described above, among others), if necessary, to fix the elongated particles in their new orientation.

Where the elongated particles are to be radially aligned with respect to a tubular medical device, an apparatus 600 like that shown in FIGS. 6A-6C may be employed. FIG. 6A is a side view of the apparatus 600, whereas FIG. 6B is a cross-sectional view taken along the plane corresponding to line b-b of FIG. 6A, while FIG. 6C is a cross-sectional view taken along the plane corresponding to line c-c of FIG. 6A. As seen from these Figures, the apparatus includes a tubular substrate, specifically a tubular medical device such as a balloon 610, to whose outer surface has been applied a liquid suspension of elongated particles 620, for example, using a technique selected from those previously discussed, among others. The elongated particles within the suspension 620 may be oriented radially by applying a suitable voltage between axial electrode A and cylindrical electrode B. As above, after solidification of the suspension 620, the particles are set in the alignment that is generated by the applied voltage.

In certain embodiments of the invention, one or more therapeutic agents may be incorporated over, within or beneath the particle containing regions.

Specific examples include, for example, therapeutic agent selected from anti-thromobotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, agents that interfere with endogenous vasoactive mechanisms, and combinations thereof, among others.

Numerous additional therapeutic agents useful for the practice of the present invention may be selected from those described in paragraphs [0040] to [0046] of commonly assigned U.S. Patent Application Pub. No. 2003/0236514, the entire disclosure of which is hereby incorporated by reference.

Some specific beneficial agents include paclitaxel, sirolimus, everolimus, tacrolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, and Serca 2 gene/protein, resiquimod, imiquimod (as well as other imidazoquinoline immune response modifiers), human apolioproteins (e.g., AI, AII, AIII, AIV, AV, etc.), vascular endothelial growth factors (e.g., VEGF-2), as well a derivatives of the forgoing, among many others.

As a specific example, a drug-delivering balloon may be made by providing a balloon with a gold plated layer (e.g., by sputtering, by electrochemical processing, or some other method), which serves as an electrode. Carbon nanotubes with thiolated end-groups (for example, formed as described in J. K. Lim et al, “Selective thiolation of single-walled carbon nanotubes,” Synthetic Metals 139 (2003) 521-527) are then attached to the gold surface. The whole assembly is moved into a cylindrical counter-electrode, and an AC field is applied between the electrode and counter-electrode to align the CNT's (which are anchored by the thiol groups) perpendicular to the gold surface. UV light is then applied to cure the polymer layer. If the CNT's are longer then the spin-coated layer is thick, they may stick out of the polymer cured layer, creating a forest of CNT needles. One may then use the gold surface underneath to drive a variety of therapeutic agents into the CNT's and subsequently, while in the body, drive the therapeutic agents out.

Where provided, the therapeutic agent need not be provided after formation of the solidified elongated particle region. For example, in certain specific embodiments, at least one therapeutic agent is added to the elongated particle suspension prior to solidification.

Although various embodiments of the invention are specifically illustrated and described herein, it will be appreciated that modifications and variations of the present invention are covered by the above teachings without departing from the spirit and intended scope of the invention.

1. A medical device comprising a particle-containing region that comprises electrically aligned, elongated particles within a matrix, wherein said medical device is configured for implantation or insertion into a subject.

2. The medical device of claim 1, wherein said medical device is selected from a balloon catheter, a graft, a stent, and a valve.

3. The medical device of claim 1, wherein at least a portion of said particle-containing region is freestanding.

4. The medical device of claim 1, wherein at least a portion of said particle-containing region is disposed on a substrate.

5. The medical device of claim 4, wherein said substrate selected from a balloon, a catheter, and a stent.

6. The medical device of claim 4, wherein said substrate selected from a metallic substrate and a polymeric substrate.

7. The medical device of claim 1, comprising a plurality of particle-containing regions.

8. The medical device of claim 1, comprising a plurality of particle-containing regions disposed laterally with respect to one another over a substrate.

9. The medical device of claim 7, comprising a first particle-containing layer disposed at least partially over a second first particle-containing layer.

10. The medical device of claim 9, wherein the elongated particles within the first layer are aligned along an axis that is perpendicular to the alignment of the elongated particles within the second layer.

11. The medical device of claim 10, wherein the first and second layers are substantially planar.

12. The medical device of claim 10, wherein the first and second layers are concentric annuli.

13. The medical device of claim 1, wherein said particle-containing region is an annular particle-containing region having an axis.

14. The medical device of claim 13, wherein the elongated particles are aligned substantially parallel to the axis of said annular particle-containing region.

15. The medical device of claim 13, wherein the elongated particles are circumferentially aligned with respect to said annular particle-containing region.

16. The medical device of claim 13, wherein the elongated particles are aligned radially with respect to an axis of said annular particle-containing region.

17. The medical device of claim 1, wherein said elongated particles have a permanent dipole.

18. The medical device of claim 1, wherein said elongated particles display an induced dipole with subjected to an electric field.

19. The medical device of claim 1, wherein said elongated particles comprise ceramic elongated particles.

20. The medical device of claim 1, wherein said elongated particles comprise conductive elongated particles.

21. The medical device of claim 1, wherein said elongated particles comprise carbon nanofilaments.

22. The medical device of claim 1, wherein said elongated particles comprise carbon nanotubes.

23. The medical device of claim 1, wherein said elongated particles comprise derivatized carbon nanotubes.

24. The medical device of claim 1, wherein said elongated particles comprise polymer-functionalized carbon nanotubes.

25. The medical device of claim 1, wherein said elongated particles comprise derivatized polyoxometallates.

26. The medical device of claim 1, wherein said matrix is a ceramic matrix.

27. The medical device of claim 26, wherein said ceramic matrix comprises a ceramic material selected from alumina, zirconia, glass-ceramics, calcium phosphate, and combinations thereof.

28. The medical device of claim 26, wherein said elongated particles comprise polyether functionalized elongated particles.

29. The medical device of claim 1, wherein said matrix is a polymeric matrix.

30. The medical device of claim 29, wherein said polymeric matrix comprises a block copolymer.

31. The medical device of claim 30, wherein said block copolymer comprises a polyalkene block and a poly(vinyl aromatic) block.

32. The medical device of claim 31, wherein said elongated particles comprise polymer-derivatized elongated particles in which the derivatizing polymer comprises a polyalkene block, a poly(vinyl aromatic) block, or both.

33. The medical device of claim 30, wherein said block copolymer comprises an ether block and a polyamide block.

34. The medical device of claim 33, wherein said elongated particles comprise polymer-derivatized elongated particles in which the derivatizing polymer comprises a polyether block, a polyamide block, or both.

35. The medical device of claim 1, wherein said particle-containing region comprises elongated particles having a first charge and charged polymers having a second charge that is opposite to that of said first charge.

36. A method of providing the medical device of claim 1, comprising:
providing a liquid suspension comprising said elongated particles;
applying an electric field to said suspension, said electric field having sufficient strength to align said elongated particles in an aligned orientation; and
fixing the elongated particles in said aligned orientation.

37. The method of claim 36, wherein said field is generated using a DC voltage.

38. The method of claim 36, wherein said field is generated using an AC voltage.

39. The method of claim 36, wherein said field is generated using an AC voltage with a DC bias.

40. The method of claim 36, wherein said liquid suspension further comprises a matrix material and wherein said liquid suspension is solidified to fix said elongated particles in said aligned orientation.

41. The method of claim 40, wherein said liquid suspension is selected from (a) a liquid suspension comprising said elongated particles in a polymer melt, (b) a liquid suspension comprising said elongated particles in a polymer solution, (c) a liquid suspension comprising said elongated particles in a curable polymeric liquid, (d) a liquid suspension comprising said elongated particles and ceramic particles.

42. The method of claim 36, wherein said elongated particles are fixed upon being electrostatically assembled on a surface of opposite charge.
